SEQUENCE LISTING (11 sequences)

SEQ ID NO: 1 - RNA, 24 nt, Homo sapiens
uucuccaaaagaaagcacuuucug

SEQ ID NO: 2 - DNA, 51 bp, Homo sapiens
tagatgactggttgcgtcatttggagaagtgagtgctccttgatggtggaa

SEQ ID NO: 3 - DNA, 8 bp, Artificial Sequence
  Polynucleotide
ttggagaa

SEQ ID NO: 4 - RNA, 8 nt, Artificial Sequence
  Polynucleotide
uucuccaa

SEQ ID NO: 5 - RNA, 83 nt, Homo sapiens
ucucaugcagucauucuccaaaagaaagcacuuucuguugucugaaagcagagugccuucuuuuggagcguuacuguuugaga

SEQ ID NO: 6 - DNA, 30 bp, Artificial Sequence
  Polynucleotide
tccctcgagcccattccagcagtcccagtt

SEQ ID NO: 7 - DNA, 37 bp, Artificial Sequence
  Polynucleotide
ataagaatgcggccgctgcccacggatgactgctgag

SEQ ID NO: 8 - DNA, 32 bp, Artificial Sequence
  Polynucleotide
ggttgcgtcatttgactaagtgagtgctcctt

SEQ ID NO: 9 - DNA, 37 bp, Artificial Sequence; identical to SEQ ID NO: 7
  Polynucleotide

SEQ ID NO: 10 - DNA, 28 bp, Artificial Sequence
  Polynucleotide
gcggatccggtacatgccaccacaggcg

SEQ ID NO: 11 - DNA, 30 bp, Artificial Sequence
  Polynucleotide
gcgaattcgcagtgagcagagaacatacca

(19) United States
(12) Patent Application Publication
(10) Pub. No.: US 2013/0131148 A1
(43) Pub. Date: May 23, 2013

(54) MICRO-RNA FOR CANCER DIAGNOSIS, PROGNOSIS AND THERAPY

(71) Applicants: Noam Shomron, Herzlia (IL); Liat Edry, Petach-Tikva (IL); Efrat Mamluk, Kibbutz Givat Haim Meuchad (IL); Eitan Friedman, Tel-Aviv (IL)

(72) Inventors: Noam Shomron, Herzlia (IL); Liat Edry, Petach-Tikva (IL); Efrat Mamluk, Kibbutz Givat Haim Meuchad (IL); Eitan Friedman, Tel-Aviv (IL)

(21) Appl. No.: 13/703,658

(22) PCT Filed: Mar. 10, 2011
(86) PCT No.: PCT/IL11/00238

§ 371 Date: Dec. 12, 2012

Related U.S. Application Data
(60) Provisional application No. 61/322,922, filed on Apr. 12, 2010.

(51) Int. Cl. C12Q 1/68; A61K 48/00; A61K 31/7088

(52) U.S. Cl. CPC C12Q 1/6886; A61K 31/7088; A61K 48/005
USPC 514/44 A; 435/6.11; 435/6.12; 514/44 R; 435/320.1; 435/375

(57) ABSTRACT

The present invention relates to polymorphic binding sites for micro-RNA and to micro-RNA-based cancer diagnosis, cancer therapy and personalized medicine. In particular, the present invention relates to diagnosis, therapy and personalized medicine with the specific miR-515-5p molecule.

### FIELD OF THE INVENTION

The present invention relates to micro-RNA (miRNA)-based cancer diagnosis, prognosis and therapy.

### BACKGROUND OF THE INVENTION

Cancer, the uncontrolled growth of abnormal cells in the body, is a major health problem worldwide and one of the leading causes of death in developed countries. The malignant transformation process is a complex process involving genetic, epigenetic and environmental factors. One group of molecules suggested to be implicated in this process is the microRNA molecules (also referred to as “miRNA”, “miR”).

MicroRNAs are small single-stranded RNA molecules, about 22 nucleotides (nt) long, that regulate gene expression. miRNAs are naturally abundant and evolutionarily conserved non-coding RNA molecules found in a variety of organisms, including plants and animals. They function as a novel class of global gene regulators by binding to partially complementary sequences in untranslated regions (UTRs) of downstream target mRNAs and triggering their repression. Currently there are hundreds of reported human miRNAs predicted to control at least half of the human transcriptome.

The biogenesis of a miRNA initiates in the nucleus by RNA polymerase II transcription, generating a primary transcript (pri-miRNA). The primary transcript is cleaved by Drosha ribonuclease III enzyme to produce an approximately 70 nt stem-loop precursor miRNA (pre-miRNA). The pre-miRNA is then actively exported to the cytoplasm where it is cleaved by Dicer ribonuclease to form the mature miRNA. One strand of this miRNA is incorporated into an RNA-induced silencing complex (RISC) which recognizes target mRNAs through imperfect base pairing with the miRNA, and most commonly results in translational inhibition or destabilization of the target mRNA. Typically, the target mRNA contains a sequence complementary to a “seed” sequence of the miRNA, which usually corresponds to nucleotides 2-8 of the miRNA. The seed sequence is considered to be essential for the binding of the miRNA to the mRNA.

In addition to their presence in cells, it has been reported that miRNAs are also present in bodily fluids, such as the serum (see for example Gilad et al. (2008) “Serum MicroRNAs are promising novel biomarkers”, PLoS One, 3(9):e3148).

miRNAs were observed to be important for a diverse range of biological processes such as development, differentiation, proliferation, growth, cell cycle and apoptosis. Their aberrant expression was suggested to be involved in many human diseases, including cancer as noted above, and indeed certain miRNAs were found to play a role in specific types of cancer. Several methods have been proposed for the diagnosis and treatment of cancer, which utilize specific miRNAs.

U.S. Patent Application Publication No. 2008/0306006 discloses miR-based methods and compositions for the diagnosis and treatment of solid cancers. Methods of identifying inhibitors of tumorigenesis are also disclosed.

U.S. Patent Application Publication No. 2008/0261908 discloses miR-based methods and compositions for the diagnosis, prognosis and treatment of breast cancer. Methods of identifying anti-breast cancer agents are also disclosed.

US Patent Application Publication No. 2007/0026405 discloses materials and methods for colorectal cancer screening, diagnosis and therapy, based on expression of Prox 1. The invention provides, inter alia, a method of inhibiting the growth of colon cancer cells in a mammalian subject by using a molecule that suppresses expression of Prox-1. The inhibiting molecules may include, inter alia, a micro-RNA that inhibits Prox-1 expression.

WO 2010/061396 discloses methods for detecting an altered susceptibility to breast and ovarian cancer in a subject carrying a BRCA mutation, comprising determining the nucleic acid sequence of a polymorphism of a microRNA-related gene.

The insulin-like growth factor 1 receptor (IGF1R) is a transmembrane receptor belonging to the large class of tyrosine kinase receptors. IGF1R is frequently overexpressed in tumors and is known to play an important role in malignant cell transformation. IGF1R is implicated in a wide variety of cancer types, including but not limited to, breast, prostate, lung, pancreas and hepatocellular carcinoma (HCC). The gene encoding IGF1R contains a single nucleotide polymorphism (SNP) identified as rs28674628, which is located in the 3′ UTR of the mRNA. WO 2010/061396 noted above discloses that this SNP is located in the binding site of miR-151-5p.

There still remains a need for more effective means of cancer diagnosis, prognosis and treatment. It would be highly beneficial to identify novel miRNA molecules involved in specific types of cancers, that may be utilized as diagnostic, prognostic and therapeutic tools.

### SUMMARY OF THE INVENTION

The present invention provides diagnostic, prognostic and therapeutic uses of a specific miRNA molecule, namely miR-515-5p having the sequence 5′-UUCUCCAAAAGAAAGCACUUUCUG-3′ (SEQ ID NO: 1).

The present invention discloses for the first time that miR-515-5p can bind to the 3′ untranslated region (3′ UTR) of the mRNA of the insulin-like growth factor 1 receptor (IGF1R), and regulate its expression.

As mentioned above, the gene encoding IGF1R contains a SNP identified as rs28674628, which is located in the 3′ UTR of the mRNA. It is now disclosed that this SNP is located within the binding site of miR-515-5p. The nucleotide present at this particular location is either A or G. As exemplified hereinbelow, miR-515-5p binds more strongly to an mRNA containing the common A allele compared to an mRNA containing the rare G allele. Therefore, an AA genotype is regulated by miR-515-5p to a greater extent compared to an AG genotype.

The present invention further discloses that decreased levels of miR-515-5p are indicative of the level of cancer risk in a subject. The levels of miR-515-5p can be measured in cells, tissues or bodily fluids, such as blood, serum or plasma. It was surprisingly found that miR-515-5p expression in breast tissues obtained from breast cancer patients is lower in the tumor tissue compared to adjacent healthy tissue, as exemplified hereinbelow. The tumor tissues were also found to have higher levels of IGF1R compared to the healthy tissues. In some embodiments, decreased serum levels of miR-515-5p are indicative of the level of cancer risk in a subject.

The present invention further discloses that manipulation of miR-515-5p levels is useful to reduce specifically the levels of IGF1R in cancer cells that harbor the AA genotype. It was surprisingly found that inducing overexpression of miR-515-5p down regulates expression of IGF1R, both on mRNA and protein levels, as exemplified hereinbelow.

According to one aspect, the present invention provides a method for cancer diagnosis, prognosis and monitoring in a subject, the method comprising:
(i) providing a biological sample from the subject;
(ii) measuring the level of miR-515-5p in the sample;
(iii) comparing the level of miR-515-5p in the sample to a control level of miR-515-5p;

wherein a significantly decreased miR-515-5p level relative to the control is indicative of the subject having cancer, or being susceptible to developing cancer, or having cancer associated with poor prognosis.

In some embodiments, the biological sample is a bodily fluid. In some embodiments, the bodily fluid is selected from the group consisting of serum, plasma and blood. Each possibility represents a separate embodiment of the invention.

In other embodiments, the biological sample is a tissue. In some embodiments, the biological sample is a tissue comprising cancer cells or suspected of harboring cancer cells.

In some embodiments, a method for cancer diagnosis in a subject is provided, the method comprising:
(i) providing a sample comprising serum from the subject;
(ii) measuring the serum level of miR-515-5p in the sample;
(iii) comparing the serum levels of miR-515-5p in the sample to a control serum level of miR-515-5p;

wherein a significantly decreased miR-515-5p serum level relative to the control is indicative of the subject having, or being susceptible to developing cancer.

In some embodiments, a method for cancer prognosis in a subject diagnosed with cancer is provided, the method comprising:
(i) providing a biological sample from the subject, the biological sample is selected from the group consisting of serum, plasma and blood;
(ii) measuring the level of miR-515-5p in the sample;
(iii) comparing the level of miR-515-5p in the sample to a control level of miR-515-5p;

wherein a significantly decreased miR-515-5p level relative to the control is indicative of poor cancer prognosis.

In some embodiments, a method for cancer prognosis in a subject diagnosed with cancer is provided, the method comprising:
(i) providing a biological sample comprising serum from the subject;
(ii) measuring the serum level of miR-515-5p in the sample;
(iii) comparing the serum levels of miR-515-5p in the sample to a control serum level of miR-515-5p;

wherein a significantly decreased miR-515-5p level relative to the control is indicative of poor cancer prognosis.

In some embodiments, a method for cancer prognosis in a subject diagnosed with cancer is provided, the method comprising:
(i) providing a biological sample comprising cancer cells from the subject;
(ii) measuring the level of miR-515-5p in the cancer cells;
(iii) comparing the levels of miR-515-5p in the cancer cells to a control level of miR-515-5p;

wherein a significantly decreased miR-515-5p level relative to the control is indicative of poor cancer prognosis.

In some embodiments, a method for monitoring cancer treatment in a subject is provided, the method comprising:
(i) providing a biological sample from the subject;
(ii) measuring the level of miR-515-5p in the sample;
(iii) comparing the resulting levels of miR-515-5p in the sample to initial levels of miR-515-5p in a sample from the same subject;

wherein an increase in the levels of miR-515-5p compared to the initial levels is indicative of a positive response to treatment.

In some embodiments, the method comprising:
(i) providing a biological sample from the subject, the biological sample is selected from the group consisting of serum, plasma and blood;
(ii) measuring the level of miR-515-5p in the sample;
(iii) comparing the resulting level of miR-515-5p in the sample to initial level of miR-515-5p in a sample from the same subject;

wherein an increase in the levels of miR-515-5p compared to the initial levels is indicative of a positive response to treatment.

In other embodiments, the method comprising:
(i) providing a biological sample comprising cancer cells from the subject;
(ii) measuring the level of miR-515-5p in the cancer cells;
(iii) comparing the resulting level of miR-515-5p in the cancer cells to initial level of miR-515-5p in cancer cells from the same subject;

wherein an increase in the serum levels of miR-515-5p compared to the initial levels is indicative of a positive response to treatment.

As used herein, the term “initial level”, when referring to a protein or RNA, refers to the cellular level of a certain protein or RNA molecule before the beginning of treatment according to the present invention.

In some embodiments, the resulting levels of miR-515-5p are compared to levels of miR-515-5p determined in a previous measurement from the same subject.

As used herein, “significantly decreased” or a “significant difference”, when referring to the level of a certain protein or miRNA, typically refers to a statistically significant difference, as can be defined by standard methods known in the art. This term typically refers to a difference measured by, for example, determining miR-515-5p serum level in a test sample from a subject suspected of having or being at risk of developing cancer, and comparing the resulting level to the miR-515-5p serum level of control, or reference, samples. Control samples in this case are from healthy subjects, not afflicted with cancer. In some embodiments, when one or more mutations are associated with the particular type of cancer to be assessed, control samples are from subjects not carrying the specific mutation or mutations. In some embodiments, samples are provided from a population of healthy subjects, and statistical methods are employed to determine a range of miR-515-5p serum level that is considered normal and/or an average miR-515-5p serum level that is considered normal.

Alternatively, “significantly decreased” or a “significant difference” may refer to a difference measured by, for example, determining miR-515-5p levels in cancer cells or tissues from a subject afflicted with cancer, and comparing the resulting level to the miR-515-5p levels in normal, non-cancerous cells or tissues from the same subject.

Typically, the diagnostic and prognostic methods of the present invention are employed for cancer types characterized by at least one of increased level of IGF1R and decreased level of miR-515-5p in at least a portion of the cancer cells compared to normal levels in non-cancerous cells.

In some embodiments, the cancer type is selected from the group consisting of breast, ovarian, prostate, lung, pancreas, hepatocellular carcinoma and colorectal. Each possibility represents a separate embodiment of the invention.

In some embodiments, the cancer type is selected from the group consisting of breast and ovarian cancer. In some specific embodiments, the cancer type is selected from the group consisting of breast and ovarian cancer, and the tested subjects are BRCA1 and/or BRCA2 mutation carriers. Each possibility represents separate embodiment of the invention.

In some embodiments, the cancer type is selected from the group consisting of breast and ovarian cancer. According to these embodiments, the control samples are obtained from subjects that bear the normal BRCA1 and BRCA2 gene rather than BRCA1 and/or BRCA2 mutation.

In some embodiments, the cancer diagnosis and/or prognosis is performed for subjects who carry an AA genotype in a polymorphic site within the IGF1R gene having a reference sequence (rs) number 28674628.

As used herein, the term “measuring a level” or “determining a level” are used interchangeably and includes determining a concentration, an expression level and/or a relative or absolute amount of a specific protein and/or miRNA in a sample.

According to yet another aspect, the present invention provides a method for treating cancer, the method comprising administering to a subject in need thereof a nucleic acid construct comprising miR-515-5p or an analogue or derivative thereof.

As used herein, an “analogue” or “derivative” of miR-515-5p refers to a variant miR-515-5p that has been modified without adversely affecting its ability to recognize and hybridize with the target area within the 3′UTR of the IGF1R mRNA. The analogue or derivative may include naturally-occurring, as well as synthetic nucleotides. The analogue or derivative may include chemical modifications. The analogue or derivative may include one or more base substitutions. The analogue or derivative generally retains the properties or activity observed in the naturally-occurring miR-515-5p to the extent that the analogue or derivative is useful for similar purposes as the naturally-occurring miR-515-5p. Thus, an analogue or derivative is a nucleic acid molecule that mimics the function of the naturally-occurring miR-515-5p within cells, i.e., capable of hybridizing with the target area within the 3′UTR of the IGF1R mRNA and mediating its destabilization and down-regulation.

As noted above, miRNAs typically down-regulate the stability and translation of target mRNAs that contain a sequence complementary to the miRNA seed region (nucleotides 2-8 of the miRNA) in their 3′UTR. As exemplified hereinbelow, the following complementary regions were identified within the IGF1R gene and miR-515-5p:

IGF1R:
(SEQ ID NO: 2)
5′-TAGATGACTGGTTGCGTCATTTGGAGAAGTGAGTGCTCCTTGATGG
TGGAA
miR-515-5p:
3′-GUCUUUCACGAAAGAAAACCUCUU-5′

The underlined sequences represent the target area within the IGF1R gene and the seed region of miR-515-5p (SEQ ID NOs: 3 and 4, respectively).

In some typical embodiments, the analogue or derivative comprises the seed sequence set forth in SEQ ID NO: 4.

Typically, cancer types suitable for treatment according to embodiments of the present invention are characterized by at least one of increased level of IGF1R and decreased level of miR-515-5p in at least a portion of the cancer cells compared to normal levels in non-cancerous cells. Without wishing to be bound by any particular mechanism or theory, it is contemplated that by elevating the levels of miR-515-5p in the cancer cells, the levels of IGF1R can be reduced and subsequent cellular events associated with cancer can be inhibited.

Typically, the nucleic acid construct is administered in an amount effective for suppression of IGF1R cellular expression. In some embodiments, the nucleic acid construct is administered in an amount effective to suppress cellular expression of IGF1R by at least 30%, at least 50%, at least 80% relative to the initial levels. Each possibility represents a separate embodiment of the invention.

In some embodiments, the cancer type is selected from the group consisting of breast, ovarian, prostate, lung, pancreas, and hepatocellular carcinoma and colorectal. Each possibility represents a separate embodiment of the invention.

In some embodiments, the cancer type is selected from the group consisting of breast and ovarian cancer. In some specific embodiments, the cancer type is selected from the group consisting of breast and ovarian cancer, and the treated patients are BRCA1 and/or BRCA2 mutation carriers. Each possibility represents separate embodiment of the invention.

In some typical embodiments, the treated patients are subjects who carry an AA genotype in the polymorphic site within the IGF1R gene designated by reference sequence (rs) number 28674628.

As used herein, “treating” and “treatment” encompass inhibition or suppression of cancer cells and tumors. In particular, the terms refer to inhibition or suppression of the proliferation of at least a portion of the cancer cells, namely, killing the cells, or permanently or temporarily arresting or slowing the growth of the cells. The terms also encompass inhibition of cell migration and invasion, thereby inhibition or suppression of the formation of metastases. The terms also encompass tumor shrinkage Inhibition of cancer cell proliferation may be inferred, for example, if the number of such cells in a subject remains constant or decreases after administration of a nucleic acid construct comprising miR-515-5p or an analogue or derivative thereof according to embodiments of the present invention.

In some embodiments, the nucleic acid construct comprises a precursor of miR-515-5p or an analogue or derivative thereof.

In some embodiments, the nucleic acid construct is formulated into a pharmaceutical composition. According to these embodiments, the method comprises administering to a patient a pharmaceutical composition comprising a nucleic acid construct comprising miR-515-5p or an analogue or derivative thereof.

In some embodiments, administration is performed near or directly into the tumor. For example, direct injection. In other embodiments, systemic administration is selected. According to these embodiments, the administered substance and/or the delivery vehicle typically comprises a targeting moiety or a mechanism for selective activity within the cancer cells only.

In principle, administration may be performed by any route of administration, including local and systemic routes. Systemic administration includes all enteral and all parenteral routes. Non-limiting examples of suitable administration routes include topical application, oral, rectal, transmucosal such as transnasal and buccal, intravenous, intramuscular, transdermal, subcutaneous, intradermal, intravitreal, intravesicular and inhalation routes.

According to another aspect, the present invention provides a method for treating cancer, the method comprising administering to a subject in need thereof a nucleic acid construct comprising a sequence encoding miR-515-5p or an analogue or derivative thereof.

Typically, a DNA sequence encoding miR-515-5p or an analogue or derivative thereof is operably linked to suitable regulation sequences enabling expression in mammalian cells. In some embodiments, the regulation sequences enable constitutive expression in mammalian cells. Accordingly, the method comprises administering a nucleic acid construct comprising a sequence encoding miR-515-5p or an analogue or derivative thereof, operably linked to a promoter suitable for expression in a mammalian cell.

In some embodiments, the nucleic acid construct comprising a sequence encoding miR-515-5p or an analogue or derivative thereof is an expression vector. According to these embodiments, the method comprises administering to a patient an expression vector comprising a sequence encoding miR-515-5p or an analogue or derivative thereof.

According to another aspect, the present invention provides a nucleic acid construct comprising miR-515-5p or an analogue or derivative thereof, for use in the treatment of cancer.

According to another aspect, the present invention provides a nucleic acid construct comprising a sequence encoding miR-515-5p or an analogue or derivative thereof, for use in the treatment of cancer.

According to another aspect, the present invention provides a composition comprising a nucleic acid construct comprising miR-515-5p or an analogue or derivative thereof, for use in the treatment of cancer.

According to yet another aspect, the present invention provides a composition comprising a nucleic acid construct comprising a sequence encoding miR-515-5p or an analogue or derivative thereof, for use in the treatment of cancer.

In some embodiments, a composition of the present invention further comprises a pharmaceutically acceptable diluent, excipient, or carrier.

The method of the present invention may be useful for personalized medicine. In some embodiments, the method comprises a step of measuring at least one parameter selected from the group consisting of the level of IGF1R and the level of miR-515-5p in cancer cells from the subject to be treated prior to the beginning of the treatment. Each possibility represents a separate embodiment of the invention. In some embodiments, an effective treatment for a subject is determined based on the initial level of miR-515-5p. In additional embodiments, an effective treatment for a subject is determined based on the initial level of IGF1R.

In some embodiments, periodical measurements of the levels of miR-515-5p and/or IGF1R in the cancer cells are performed. Such measurements may be useful in evaluating the efficiency of the treatment and optionally adjusting the treatment plan.

Thus, according to another aspect, the present invention provides a method of personalized medicine for cancer, the method comprising measuring cellular levels of miR-515-5p in cancer cells from a subject having a particular cancer, and proposing a treatment with a likelihood of being effective for said subject based on the cellular levels of miR-515-5p in said cancer cells. It is contemplated that the levels of miR-515-5p in a subject affect inter-individual response to treatments.

According to another aspect, the present invention provides a method of inhibiting cancer cells proliferation comprising manipulating the levels of miR-515-5p in the cancer cells.

In some typical embodiments, over-expression of miR-515-5p is induced. In some embodiments, the method comprises administering a nucleic acid construct comprising miR-515-5p or an analogue or derivative thereof. In some embodiments, the method comprises administering a nucleic acid construct encoding a sequence comprising miR-515-5p or an analogue or derivative thereof. In some embodiments, an expression vector comprising a nucleic acid construct encoding a sequence comprising miR-515-5p or an analogue or derivative thereof is administered.

In some embodiments, the cancer cells harbor an AA genotype in the polymorphic site within the IGF1R gene designated by reference sequence (rs) number 28674628.

These and further aspects and features of the present invention will become apparent from the figures, the detailed description, examples and claims which follow.

### BRIEF DESCRIPTION OF THE FIGURES

FIG. 1. Kaplan-Meier analysis of the breast cancer appearance (survival), among Jewish women who carry BRCA1 mutations, with the genotype of rs28674628. Homozygote (AA), n=157 is shown on the right; Heterozygote (GA), n=10 is shown on the left.

FIG. 2. miR-515-5p over-expression down-regulates IGF1R homozygote (AA) form. Assessment of Luciferase reporter activity in HEK 293T cells cotransfected with miR-515-5p in combination with either the Renilla/firefly Luciferase psiCHECK2 control, the psiCHECK2 Luciferase constructs of 3′UTR IGF1R homozygote (AA), heterozygote (GA) and knockdown control (ACT). The data presented is the relative expression level of Firefly Luciferase expression standardized Renilla Luciferase.

FIG. 3. miR-515-5p over-expression down regulates IGF1R. A) In the mRNA level. miR-515-5p over-expression in HeLa cells can down regulate endogenous levels of IGF1R mRNA versus BRCA. The data were analyzed with SDS software (ABI) and the RQ (relative quantity) Manager Software, for automated data analysis and were calculated based on the comparative threshold cycle (Ct) method. B). In the protein level. Western Blot analysis of IGF1R and Actin 1 in HeLa cells constitutively expressing miR-515-5p. The quantification of the bands was done using ImageJ software and the relative expression of IGF1R is indicated below in percent.

FIG. 4. A) Immunostaining for IGF1R in breast tissues obtained from breast cancer patients BRCA1/BRCA2 mutation carriers. Cytoplasmic positive immunoexpression of IGF1R is found in carcinoma cells in contrast to negative non-neoplastic surrounding tissue. B) Quantitative RT-PCR analysis of miR-515-5p expression level. RNA was extracted from paraformaldehyde-fixed paraffin-embedded (FFPE) normal (Av-Average) and cancerous tissues from four BRCA1/BRCA2 mutation carriers. The data were normalized to U6 snRNA and analyzed with SDS software (ABI). The comparative threshold cycle (Ct) method was used for quantitation.

### DETAILED DESCRIPTION OF THE INVENTION

The present invention provides diagnostic, prognostic and therapeutic uses of a specific miRNA molecule, namely miR-515-5p having the sequence 5′-UUCUCCAAAAGAAAGCACUUUCUG-3′ (SEQ ID NO: 1). This sequence corresponds to the mature miRNA molecule. Information concerning miRNAs and associated pri-miRNA and pre-miRNA sequences is available in miRNA databases such as miRBase (Griffiths-Jones et al. 2008 Nucl Acids Res 36, (Database Issue: D154-D158) and the NCBI human genome database.

The term “miR-515-5p” typically refers to“hsa-miR-515-5p”, which is the human micro-RNA 515-5p with the sequence set forth in SEQ ID NO: 1.

### DEFINITIONS

As used herein, the term “gene” has its meaning as understood in the art. In general, a gene is taken to include gene regulatory sequences (e.g. promoters, enhancers, etc.) and/or intron sequences, in addition to coding sequences (open reading frames).

As used herein, the term “isolated” means 1) separated from at least some of the components with which it is usually associated in nature; 2) prepared or purified by a process that involves the hand of man; and/or 3) not occurring in nature.

The terms “polypeptide,” “peptide” and “protein” are used interchangeably herein to refer to a polymer of amino acid residues.

As used herein, the term “construct”, when referring to nucleic acids, refers to an artificially assembled or isolated nucleic acid molecule which comprises the gene of interest.

As used herein, the term “vector” refers to any recombinant polynucleotide construct that may be used for the purpose of transformation or transfection, i.e. the introduction of heterologous DNA into a cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced.

As used herein, the term “expression vector” refers to a vector containing a nucleic acid sequence coding for at least part of a gene product capable of being transcribed. Expression vectors typically contain a variety of “control sequences,” which refer to nucleic acid sequences necessary for the transcription and translation of an operably linked coding sequence in a particular host organism. In addition to control sequences that govern transcription and translation, vectors and expression vectors may contain nucleic acid sequences that serve other functions as well.

As used herein, a “primer” defines an oligonucleotide which is capable of annealing to (hybridizing with) a target sequence, thereby creating a double stranded region which can serve as an initiation point for DNA synthesis under suitable conditions.

As used herein, the terms “transformation” and “transfection” refer to the introduction of foreign DNA into cells. Transformation or transfection may be “stable”, where the introduced DNA is incorporated into the genome of the cell, or “transient”, where the introduced DNA is not incorporated into the genome of the cell. The terms “transformants” or “transformed cells” or “transfected cells” include the primary transformed or transfected cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

The terms “polynucleotide”, “polynucleotide sequence”, “nucleic acid”, “nucleic acid sequence” and “oligonucleotide” are used interchangeably herein to refer to polymeric forms of nucleotides of any length, such as deoxyribonucleotides, ribonucleotides, or modified forms thereof in the form of an individual fragment or as a component of a larger construct, in a single strand or in a double strand or multi-strand form. The terms encompass sense and antisense sequences of DNA or RNA, genomic DNA, cDNA, DNA-RNA hybrids, or a polymer comprising purine and pyrimidine bases or other natural, chemically or biochemically modified, non-natural, or derivatized nucleotide bases. Further included are mRNA or cDNA that comprise intronic sequences. The backbone of the polynucleotide can comprise sugars and phosphate groups (as typically found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer (see, e.g., Peyrottes et al. (1996) Nucl. Acids Res. 24:1841-1848; Chaturvedi et al (1996) Nucl. Acids Res. 24:2318-2323). A polynucleotide may comprise modified nucleotides, such as methylated nucleotides and nucleotide analogs, uracyl, other sugars, and linking groups such as fluororibose and thioate, and nucleotide branches. The sequence of nucleotides may be interrupted by non-nucleotide components. A polynucleotide may be further modified after polymerization, such as by conjugation with a labeling component, capping, substitution of one or more of naturally occurring nucleotides with an analog, and introduction of means for attaching the polynucleotide to proteins, metal ions, labeling components, other polynucleotides, or a solid support.

A polynucleotide may comprise a nucleotide sequence disclosed herein wherein thymidine (T) can also be uracil (U); this definition pertains to the differences between the chemical structures of DNA and RNA, in particular the observation that one of the four major bases in RNA is uracil (U) instead of thymidine (T).

As used herein, the term “operably linked” refers to the structural and functional relationship between two or more nucleic acid sequences joined together as part of the same polynucleotide, generally a regulatory sequence and a protein coding sequence or a miR coding sequence. Non-limiting examples of regulatory sequences include a promoter, enhancer, ribosome binding site. Generally, “operably linked” means that the nucleic acid sequences being linked are contiguous, although enhancers do not have to be contiguous. Linking is accomplished by ligation at convenient restriction sites, or if such sites do not exist, synthetic oligonucleotide adaptors or linkers may be used in accordance with conventional practice.

Transcription control sequences are sequences, which control the initiation, elongation, and termination of transcription. Particularly important transcription control sequences are those which control transcription initiation, such as promoter, enhancer, operator and repressor sequences.

As used herein in the context of polynucleotides, the terms “hybridize”, “hybridizing”, “hybridizes” and the like, refer to conventional hybridization conditions.

As used herein, the term “pharmaceutical composition” refers to a preparation comprising one or more pharmaceutically active ingredients, for example, a nucleic acid construct of the present invention, and generally further comprising at least one pharmaceutically acceptable diluent, carrier or excipient. The purpose of a pharmaceutical composition is to facilitate administration of the active ingredient to a subject.

As used herein, the term “active ingredient” refers to a component of a pharmaceutical composition that provides the primary pharmaceutical benefit, as opposed to an “inactive ingredient” which is generally recognized as providing no pharmaceutical benefit.

As used herein, the term “carrier, excipient or diluent” refers to an inactive ingredient, for example a tonicity adjusting agent, wetting agent or preservative, which facilitates formulation and/or administration of an active pharmaceutical ingredient. Such carriers, excipients and diluents are well known in the art.

As used herein, the term “pharmaceutically acceptable” refers to a non-toxic and inert substance that is physiologically compatible with humans or other mammals.

As used herein, the term “subject” refers to any mammal of any age, in particular a human subject, but also including non-human mammals.

The terms “cancer” and “neoplasm” are recognized by those skilled in the art and used herein interchangeably to refer to a disease state characterized by cells in an abnormal state or condition characterized by rapid proliferation. A “tumor” containing such cells may be benign, premalignant or malignant. The terms include disease states characterized by all types of hyperproliferative growth, hyperplastic growth, cancerous growths, oncogenic processes, metastatic tissues, malignantly transformed cells, tissues, or organs, irrespective of histopathologic type or stage of invasiveness.

The term “carcinoma” is recognized by those skilled in the art and refers to malignancies of epithelial or endocrine tissues including but not limited to respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of supportive tissues or connective tissue, for example bone or cartilage.

As used herein, the term “leukemia” refers to all cancers of the hematopoietic and immune systems (blood and lymphatic system). These terms refer to a progressive, malignant disease of the blood-forming organs, marked by distorted proliferation and development of leukocytes and their precursors in the blood and bone marrow. Myelomas refer to other types of tumors of the blood, bone marrow cells. Lymphomas refer to tumors of the lymph tissue.

Cancer Diagnosis and Prognosis

The present invention discloses an association of miR-515-5p expression levels with cancer risk and cancer prognosis. It is now disclosed that decreased levels of miR-515-5p expression are indicative of the level of cancer risk in a subject, and indicative of poor cancer prognosis. Poor prognosis may include, but is not limited to, low survival rate and rapid disease progression.

The levels of miR-515-5p may be measured in cells or tissues from a tested individual, or in a sample comprising serum from that individual (such as blood, plasma, serum).

According to one aspect, the present invention provides a method for cancer diagnosis, prognosis or monitoring in a subject, the method comprising:
(i) providing a biological sample from the subject;
(ii) measuring the level of miR-515-5p in the sample;
(iii) comparing the levels of miR-515-5p in the sample to control levels of miR-515-5p;

wherein a significantly decreased miR-515-5p level relative to the control is indicative of the subject having cancer, or being susceptible to developing cancer, or having cancer associated with poor prognosis.

In some embodiments, the biological sample is a bodily fluid. In some embodiments, the bodily fluid is selected from the group consisting of serum, plasma and blood. Each possibility represents a separate embodiment of the invention.

In other embodiments, the biological sample is a tissue. In some embodiments, the biological sample is a tissue comprising cancer cells.

As used herein, the terms “susceptibility to developing cancer” or “being at a risk of developing cancer” refer to the probability of individuals to be diagnosed with cancer.

In some embodiments, a decreased miR-515-5p level is indicative of a subject having caner. In other embodiments, a decreased miR-515-5p level is indicative of a subject susceptible of developing cancer. In some embodiments, a decreased level is indicative of poor cancer prognosis.

In some embodiments, a method for determining the cancer risk in a subject is provided, the method comprising:
(i) providing a biological sample from the subject;
(ii) measuring the level of miR-515-5p in the sample;
(iii) comparing the levels of miR-515-5p in the sample to control levels of miR-515-5p;

wherein a significantly decreased miR-515-5p level relative to the control is indicative of the subject having an increased risk of developing cancer.

In some embodiments, a method for cancer diagnosis in a subject is provided, the method comprising:
(i) providing a sample comprising serum from the subject;
(ii) measuring the serum level of miR-515-5p in the sample;
(iii) comparing the serum levels of miR-515-5p in the sample to control serum levels of miR-515-5p;

wherein a significantly decreased miR-515-5p serum level relative to the control is indicative of the subject having, or being susceptible to developing cancer.

In some embodiments, a method for cancer prognosis in a subject is provided, the method comprising:
(i) providing a biological sample from the subject, the biological sample is selected from the group consisting of serum, plasma and blood;
(ii) measuring the level of miR-515-5p in the sample;
(iii) comparing the levels of miR-515-5p in the sample to control levels of miR-515-5p;

wherein a significantly decreased miR-515-5p level relative to the control is indicative of poor cancer prognosis.

In some embodiments, a method for cancer prognosis in a subject diagnosed with cancer is provided, the method comprising:
(i) providing a biological sample comprising serum from the subject;
(ii) measuring the serum level of miR-515-5p in the sample;
(iii) comparing the serum levels of miR-515-5p in the sample to control serum levels of miR-515-5p;

wherein a significantly decreased miR-515-5p serum level relative to the control is indicative of poor cancer prognosis.

In some embodiments, a method for cancer prognosis in a subject diagnosed with cancer is provided, the method comprising:
(i) providing a biological sample comprising cancer cells from the subject;
(ii) measuring the level of miR-515-5p in the cancer cells;
(iii) comparing the levels of miR-515-5p in the cancer cells to control levels of miR-515-5p;

wherein a significantly decreased miR-515-5p level relative to the control is indicative of poor cancer prognosis.

In some embodiments, the level of miR-515-5p expression in a tested tissue/sample are compared to the levels of expression in a healthy tissue/sample and/or to a basal level of expression, and a decreased level of miR-515-5p correlates with poor cancer prognosis.

In some embodiments, a method for monitoring cancer treatment in a subject is provided, the method comprising:
(i) providing a biological sample s from the subject;
(ii) measuring the level of miR-515-5p in the sample;
(iii) comparing the resulting levels of miR-515-5p in the sample to initial levels of miR-515-5p in a sample from the same subject;

wherein an increase in the levels of miR-515-5p compared to the initial levels is indicative of a positive response to treatment.

In some embodiments, the biological sample is selected from the group consisting of blood, plasma and serum. In some particular embodiments, the biological sample is serum.

In other embodiments, the biological sample is a tissue comprising cancer cells.

As used herein, the term “initial level”, when referring to a protein or RNA, refers to the cellular level of a certain protein or RNA molecule before the beginning of treatment according to the present invention

In some embodiments, the resulting levels of miR-515-5p in a certain measurement are compared to levels of miR-515-5p determined in a previous measurement from the same subject.

The methods of the present invention comprise determining, in a sample comprising cancer cells, or alternatively in a sample comprising a bodily fluid, such as serum, the level of miR-515-5p. Methods for quantification of RNA levels are well known in the art. Non-limiting examples of such procedures include real-time PCR, microarrays, bead based arrays or any other related technology. Exemplary procedures are provided below.

MicroRNA profiling may be used to measure the levels of miR-515-5p in the cell at a given time. A non-limiting, exemplary procedure includes the following steps: Total RNA is isolated using Trizol® (Invitrogen) or RNA extraction from paraformaldehyde-fixed paraffin-embedded (NNPE) tissues using The RecoverAll™ Total Nucleic Acid Isolation Kit (Applied Biosystems). Quantitative (real-time) reverse transcriptase PCR is performed using TaqMan® MicroRNA Assay kits according to manufacturer's protocol (Applied Biosystems). PCR amplification and reading can be carried out using the ABI Prism® 7900HT Sequence Detection System under the following thermal cycler conditions: 2 min at 50° C. and 10 min at 95° C. for 40 cycles (30 s at 95° C. and 1 min at 60° C.). Also see Mor et al., Nuc Acids Res, Jan. 18, 2011.

Typically, the diagnostic and prognostic methods of the present invention are employed for cancer types characterized, inter alia, by at least one of increased level of IGF1R and decreased level of miR-515-5p in at least a portion of the cancer cells compared to normal levels in non-cancerous cells. Each possibility represents a separate embodiment of the invention.

The cancer may be a sarcoma, a carcinoma, an adenocarcinoma, a lymphoma, and a leukemia. Non-limiting examples of cancer types include breast, ovarian, prostate, lung, pancreas, hepatocellular carcinoma, colorectal, colon, cervical, endometrioid.

In some embodiments, the cancer is selected from the group consisting of breast, ovarian, prostate, lung, pancreas, hepatocellular carcinoma and colorectal. Each possibility represents a separate embodiment of the invention.

In some embodiments, the cancer is selected from the group consisting of breast and ovarian cancer. In some specific embodiments, the cancer type is selected from the group consisting of breast and ovarian cancer, and the tested subjects carry at least one mutation selected from the group consisting of BRCA1 mutation and BRCA2 mutation. Each possibility represents separate embodiment of the invention.

BRCA1 and BRCA2 are large genes in which multiple different loss-of-function mutations have been detected. BRCA1 and BRCA2 have important roles in the maintenance of genomic stability by facilitating repair of DNA double strand breaks. Several BRCA1 and BRCA2 breast and ovarian cancer predisposition mutations were identified. These mutations significantly increase lifetime risk for developing breast cancer in carrier women from about 12% (the average lifetime risk) to about 80%, and the lifetime risk for ovarian cancer from about 1.5% to about 50%. Known BRCA1 cancer predisposition mutations include 185delAG, 187delAG, 5385insC and 5382insC. Known BRCA2 cancer predisposition mutations include 6174delT and 999del5. Information about the cancer-associated mutations identified in BRCA1/2 genes can be found, for example, in GeneReviews: BRCA1 and BRCA2 Hereditary Breast and Ovarian Cancer, Petrucelli N, Daly MB, Feldman G L. 1998 Sep. 4 (updated 2011 Jan. 20), and U.S. Pat. No. 5,747,282.

Penetrance of germline BRCA1/BRCA2 mutations is incomplete and coupled with an observed differences in age at cancer diagnosis among carriers of identical mutations, suggesting that other factors—including environmental and genetic—are involved in determining mutant allele penetrance

As noted above, the nucleotide found in the polymorphic site within the IGF1R gene having a reference sequence (rs) number 28674628 may be either an A (Adenine) or G (Guanine) nucleotide. In some embodiments, the cancer diagnosis and/or prognosis is performed in subjects who carry an AA genotype in this polymorphic site. In other embodiments, cancer diagnosis and/or prognosis is performed in subjects who carry a genotype other than AA in this polymorphic site, such as AG or GG.

For assay of the genomic DNA, virtually any biological sample (other than pure red blood cells) is suitable. For example, convenient tissue samples include whole blood, semen, saliva, tears, urine, fecal material, sweat, buccal, skin and hair. Typically, a sample obtained from a subject is processed before the detecting step, e.g. the DNA in the cell or tissue is separated from other components of the sample, and the target DNA is amplified. All samples obtained from a subject, including those subjected to any sort of further processing, are considered to be obtained from the subject.

Methods for DNA amplification are known to a person skilled in the art. Most commonly used method for DNA amplification is PCR (polymerase chain reaction; see, or example, PCR Protocols: A Guide to Methods and Applications eds.).

Sequencing of the target DNA is required in order to determine the nucleotide identity in the polymorphic site. A number of methods well-known in the art can be used to carry out the sequencing reactions. For example, sequencing based on the MALDI-TOF platform can be performed by using the Sequenom iPLEX™ technology.

In some embodiments, cancer diagnosis or prognosis is performed based on serum levels of miR-515-5p in samples from the tested subject. According to these embodiments, control samples are from healthy subjects known to have only normal cells or non-cancerous cells. When one or more mutations are associated with the particular type of cancer to be assessed, control samples are typically from subjects that do not carry the specific mutation or mutations. For example, if the type of cancer is breast or ovarian cancer, the control samples are typically from subjects that do not carry BRCA1 and/or BRCA2 mutation.

In some embodiments, computer algorithms are used for analysis of samples from healthy subjects and cancer patients, in order to determine a range of miR-515-5p level that is considered normal.

In other embodiments, cancer prognosis is performed based on cellular levels of miR-515-5p in cancer cells from the tested subject. According to these embodiments, the control sample may be from non-cancerous cells or tissues of the same subject. Alternatively, the control sample may be from non-cancerous cells or tissues of a healthy subject, not afflicted with cancer.

Cancer Treatment

The present invention discloses that miR-515-5p binds to a SNP that resides in the 3′ UTR of the gene encoding IGF1R. The effect of the polymorphism (i.e. the identity of the nucleotide in that position) on miR-515-5p binding to that site was tested experimentally using a reporter gene assay, as exemplified hereinbelow. It was found that the AA homozygous form (that was associated with better prognosis) is strongly regulated by miR-515-5p. In addition, the influence of the polymorphism on the levels of IGF1R expression was assayed directly, as also exemplified hereinbelow. It was observed that miR-515-5p can regulate IGF1R levels via the polymorphic site in its 3′ UTR. Specifically, it was shown that over-expression of miR-515-5p can reduce the translation of IGF1R.

Thus, manipulation of the levels of miR-515-5p in cancer cells can be used to inhibit their proliferation. It is contemplated that the levels of miR-515-5p in cells can control the levels of IGF1R in those cells, and thus control subsequent cellular network of events associated with cancer. Specifically, induction of miR-515-5p over-expression in cancer cells can be used to inhibit IGF1R expression in those cells. In some embodiments, the cells harbor an AA genotype in the relevant SNP.

Furthermore, manipulating the levels of miR-515-5p in cancer cells can be used as a therapeutic tool in the treatment of cancer. Specifically, administering to a patient a composition comprising a polynucleotide comprising the nucleotide sequence of miR-515-5p, i.e. GUCUUUCACGAAAGAAAAACCUCUU, can reduce the level of IGF1R in the cancer cells and inhibit subsequent cellular events associated with cancer.

According to another aspect, the present invention provides a method for treating cancer, the method comprising administering to a subject in need thereof an isolated nucleic acid construct comprising miR-515-5p or an analogue or derivative thereof.

In some embodiments, the method comprising administering an isolated nucleic acid construct comprising miR-515-5p. In some embodiments, the method comprising administering an isolated nucleic acid construct consisting of the sequence 5′-UUCUCCAAAAGAAAGCACUUUCUG-3′.

In some embodiments, the nucleic acid construct comprises a precursor of miR-515-5p or an analogue or derivative thereof. In some embodiments, the nucleic acid construct comprises the pre-miRNA of miR-515-5p. In other embodiments, the nucleic acid construct comprises the pri-miRNA of miR-515-5p. A non-limiting example of a precursor sequence is set forth in SEQ ID NO: 5. In some embodiments, the method comprising administering an isolated nucleic acid construct consisting of the sequence set forth in SEQ ID NO:5.

According to another aspect, the present invention provides a method for treating cancer, the method comprising administering to a subject in need thereof an isolated nucleic acid construct comprising a DNA sequence encoding miR-515-5p or an analogue or derivative thereof.

In some embodiments, the isolated nucleic acid construct comprises a sequence encoding miR-515-5p. In some embodiments, the isolated nucleic acid construct comprises a sequence encoding the sequence 5′-UUCUCCAAAAGAAAGCACUUUCUG-3′.

In some embodiments, the nucleic acid construct comprises a sequence encoding a precursor of miR-515-5p or an analogue or derivative thereof. In some embodiments, the nucleic acid construct comprises a sequence encoding the pre-miRNA of miR-515-5p. In other embodiments, the nucleic acid construct comprises a sequence encoding the pri-miRNA of miR-515-5p.

Typically, the sequence encoding miR-515-5p or an analogue or derivative thereof is operably linked to suitable regulation sequences enabling expression in mammalian cells. In some embodiments, the regulation sequences enable constitutive expression in mammalian cells. Accordingly, the method comprises administering a nucleic acid construct comprising a sequence encoding miR-515-5p or an analogue or derivative thereof, operably linked to regulation sequences suitable for expression in a mammalian cell.

In some embodiments, the nucleic acid construct comprising a sequence encoding miR-515-5p or an analogue or derivative thereof is an expression vector. According to these embodiments, the method comprises administering to a patient an expression vector comprising a sequence encoding miR-515-5p or an analogue or derivative thereof.

According to another aspect, the present invention provides a nucleic acid construct comprising miR-515-5p or an analogue or derivative thereof, for use in the treatment of cancer.

According to another aspect, the present invention provides a nucleic acid construct comprising a sequence encoding miR-515-5p or an analogue or derivative thereof, for use in the treatment of cancer.

According to another aspect, the present invention provides a composition comprising a nucleic acid construct comprising miR-515-5p or an analogue or derivative thereof, for use in the treatment of cancer.

According to yet another aspect, the present invention provides a composition comprising a nucleic acid construct comprising a sequence encoding miR-515-5p or an analogue or derivative thereof, for use in the treatment of cancer.

In some embodiments, the nucleic acid construct is formulated into a pharmaceutical composition. According to these embodiments, the method comprises administering to a patient a pharmaceutical composition comprising a nucleic acid construct comprising miR-515-5p or an analogue or derivative thereof.

In some embodiments, a composition of the present invention further comprises a pharmaceutically acceptable diluent, excipient, or carrier.

In some embodiments, the method further comprises a step of determining the level of miR-515-5p or an analogue or derivative thereof in a biological sample from the subject. In some embodiments, the method further comprises a step of determining the level of IGF1R in a biological sample, for example cells or tissue, from the subject.

Typically, cancer types suitable for treatment according to embodiments of the present invention are characterized by at least one of increased level of IGF1R and decreased level of miR-515-5p in at least a portion of the cancer cells compared to normal levels in non-cancerous cells. The cancer may be a sarcoma, a carcinoma, an adenocarcinoma, a lymphoma, and a leukemia. Non-limiting examples of cancer types include breast, ovarian, prostate, lung, pancreas, hepatocellular carcinoma, colorectal, colon, cervical, endometrioid.

Typically, the nucleic acid construct is administered in an amount effective for suppression of IGF1R cellular expression. In some embodiments, the nucleic acid construct is administered in an amount effective to suppress cellular expression of IGF1R by at least 30%, at least 50%, at least 70%, at least 80% relative to the initial levels. Each possibility represents a separate embodiment of the invention.

In some embodiments, the cancer type is selected from the group consisting of breast, ovarian, prostate, lung, pancreas, hepatocellular carcinoma and colorectal. Each possibility represents a separate embodiment of the invention.

In some embodiments, the cancer type is selected from the group consisting of breast and ovarian cancer. In some specific embodiments, the cancer type is selected from the group consisting of breast and ovarian cancer, and the treated patients are BRCA1 and/or BRCA2 mutation carriers. Each possibility represents separate embodiment of the invention.

In some typical embodiments, the treated patients are subjects who carry an AA genotype in the polymorphic site within the IGF1R gene designated by reference sequence (rs) number 28674628.

The methods of the present invention may be useful for personalized medicine. For example, in some embodiments, the method comprises a step of measuring at least one parameter selected from the group consisting of the level of IGF1R and the level of miR-515-5p in cancer cells obtained from the subject to be treated prior to the beginning of the treatment. Each possibility represents a separate embodiment of the invention. In some embodiments, an effective treatment for a subject is determined based on the initial level of miR-515-5p. In additional embodiments, an effective treatment for a subject is determined based on the initial level of IGF1R.

In some embodiments, periodical measurements of the levels of miR-515-5p and/or IGF1R in the cancer cells are performed. As a non-limiting example, measurements can be performed after one month, after six months, after one year etc. Such measurements may be useful in evaluating the efficiency of the treatment and optionally adjusting the treatment plan.

Thus, according to another aspect, the present invention provides a method of personalized medicine for cancer, the method comprising measuring cellular levels of miR-515-5p in cancer cells obtained from a subject having a particular cancer, and determining an effective treatment for said subject based on the cellular levels of miR-515-5p in the cancer cells. It is contemplated that the levels of miR-515-5p in a subject affect inter-individual response to treatments.

According to another aspect, the present invention provides a method of inhibiting cancer cells proliferation comprising manipulating the levels of miR-515-5p in the cancer cells.

In some typical embodiments, over-expression of miR-515-5p is induced. In some embodiments, the method comprises administering a nucleic acid construct comprising miR-515-5p or an analogue or derivative thereof. In some embodiments, the method comprises administering a nucleic acid construct encoding a sequence comprising miR-515-5p or an analogue or derivative thereof. In some embodiments, an expression vector comprising a nucleic acid construct encoding a sequence comprising miR-515-5p or an analogue or derivative thereof is administered.

In some embodiments, the cancer cells harbor an AA genotype in the polymorphic site within the IGF1R gene designated by reference sequence (rs) number 28674628.

The constructs, vectors and nucleic acid sequences of the present invention may be produced using standard recombinant, enzymatic and chemical synthetic methods well known in the art. A combination of such techniques may be used.

An isolated nucleic acid sequence may be obtained from its natural source, either as an entire (i.e., complete) gene or a portion thereof. The desired nucleic acid molecule may be produced by polymerase chain reaction (PCR) amplification from a genomic template using suitable primer sequences. The amplified fragment may be manipulated by addition of synthetic restriction endonuclease sites, linkers etc., for cloning into a particular vector enabling propagation and/or further manipulation and/or expression of a particular nucleic acid sequence. Such procedures enable construction of a single molecule e.g. construct or vector, characterized by the juxtaposition of specific heterologous nucleic acid sequences and including suitable flanking sequences which enable the desired functionalities of the construct. In addition, the desired nucleic acid molecules may be produced by assembly of chemically synthesized oligonucleotides (see e.g. Sambrook et al., 2001, Molecular Cloning: A Laboratory Manual; Ausubel et al., supra). For example, a nucleic acid sequence produced by oligonucleotide assembly may be ligated to a heterologous sequence that was produced by PCR amplification, and the resultant molecule may be manipulated by addition of synthetic restriction endonuclease sites, linkers etc., for cloning into a particular expression vector, as is well known in the art.

Synthetic methods suitable for preparing nucleic acid molecules are well known in the art and include for example, assembly of oligonucleotides (see e.g. U.S. Pat. No. 5,583,013), or in vitro chemical synthesis using phosphotriester, phosphite or phosphoramidite chemistry and solid phase techniques such as those described in EP 266,032, or via deoxynucleoside H-phosphonate intermediates (see e.g. U.S. Pat. No. 5,705,629). Various different mechanisms of oligonucleotide synthesis have been disclosed in for example, U.S. Pat. Nos. 4,659,774; 4,816,571; 5,141,813; 5,264,566; 4,959,463; 5,428,148; 5,554,744; 5,574,146, and 5,602,244.

Alternatively, RNA molecules may be generated by in vitro and in vivo transcription of DNA sequences encoding the micro-RNA molecule. Such DNA sequences may be incorporated into a wide variety of vectors which incorporate suitable RNA polymerase promoters such as the T7 or SP6 polymerase promoters. Alternatively, cDNA constructs that synthesize the micro-RNA constitutively or inducibly, depending on the promoter used, can be introduced stably or transiently into cells.

Methods for achieving over-expression of miR-515-5p in cells are well known to a person of skill in the art and include, but are not limited to introducing into a cell a nucleic acid molecule encoding miR-515-5p in order to up-regulate the capacity of said cell to express miR-515-5p.

The nucleic acid constructs of the invention may be introduced into a cell by any method known to those skilled in the art.

In some embodiments, delivery vehicles are used, including but not limited to, viral vectors, liposomes, polymers, microspheres, gene therapy vectors, naked DNA vectors, carbon nanotubes and chemical linkers. Additional non-limiting examples include dendrimers, biodegradable polymers, polymers of amino acids, polymers of sugars, and oligonucleotide-binding nanoparticles. In addition, pluoronic gel as a depot reservoir can be used to deliver oligonucleotide molecules over a prolonged period. The above methods are described in, for example, Hughes et al., Drug Discovery Today 6, 303-315 (2001); Liang et al. Eur. J. Biochem 269 5753-5758 (2002); and Becker et al., In Antisense Technology in the Central 14 Nervous System (Leslie, R. A., Hunter, A. J. & Robertson, H. A., eds), pp. 147-157, Oxford University Press.

In some embodiments, administration is performed near or directly into the tumor. For example, direct injection. In other embodiments, systemic administration is selected. According to these embodiments, the administered construct and/or the delivery vehicle typically comprises a targeting moiety or a mechanism for selective activity within the cancer cells only.

In principle, administration may be performed by any route of administration, including local and systemic routes. Systemic administration includes all enteral and all parenteral routes. Non-limiting examples of suitable administration routes include topical application, oral, rectal, transmucosal such as transnasal and buccal, intravenous, intramuscular, transdermal, subcutaneous, intradermal, intravitreal, intravesicular and inhalation routes.

Targeting the nucleic acid constructs to a particular cell can be performed by any method known to those skilled in the art. For example, the construct can be conjugated to an antibody that recognizes cell surface antigens unique to cancer cells, or that are more prevalent on cancer cells, compared to normal cells. As another example, the construct can be conjugated to a ligand specifically recognized by receptors unique to cancer cells, or that are more prevalent on cancer cells. In some exemplary embodiments, a targeting moiety can be selected such that the construct is delivered to cancer cells over-expressing the IGF1 receptor.

The effective amount of the nucleic acid construct to be administered can be determined during pre-clinical trials and clinical trials by methods familiar to physicians and clinicians.

Pharmaceutical compositions of the present invention may be formulated in conventional manner using one or more pharmaceutically acceptable carriers, diluents, excipients or auxiliaries which facilitate processing of the nucleic acids into preparations which can be used pharmaceutically. Proper formulation is dependent upon the route of administration chosen.

Non-limiting examples of carriers include starch, certain types of clay, gelatin, stearic acid or salts thereof, magnesium or calcium stearate, talc, vegetable fats or oils, gums and glycols. The pharmaceutical carrier may also comprise one or more of the following: a stabilizer, a surfactant, preferably a nonionic surfactant, and optionally a salt and/or a buffering agent.

In some embodiments, the compositions of the present invention are formulated for oral administration. Non-limiting examples of formulations for oral administration include tablets, pills, dragees, capsules, liquids, gels, syrups, slurries, suspensions, and the like, for oral ingestion by a patient. Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

For administration by injection, the active ingredients of the composition may be formulated in aqueous solutions, for example in physiologically compatible buffers including but not limited to Hank's solution, Ringer's solution, or physiological salt buffer. Formulations for injection may be presented in unit dosage forms, for example, in ampoules, or in multi-dose containers with, optionally, an added preservative. The compositions may be in the form of suspensions, solutions, or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions. Alternatively, the active ingredient may be in powder form for constitution with a suitable vehicle, for example, a sterile, pyrogen-free, water-based solution, before use.

For transmucosal administration, penetrants appropriate to the barrier to be permeated are used in the formulation. Such penetrants are generally known in the art. For buccal administration, the compositions may take the form of tablets or lozenges formulated in conventional manner.

For administration by inhalation route, the active ingredients are conveniently delivered in the form of an aerosol spray presentation from a pressurized pack or a nebulizer with the use of a suitable propellant, for example, dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

In some embodiments, the compositions of the present invention are formulated for rectal administration, for example, as suppositories or retention enemas, using, for example, conventional suppository bases such as cocoa butter or other glycerides.

In some embodiments, the compositions of the present invention are formulated for topical use. Non-limiting examples of formulations for topical use include cream, ointment, lotion, gel, foam, suspension, aqueous or cosolvent solutions, salve, liposome and sprayable liquid form. The composition may also form part of a patch for transdermal application.

The nucleic acids may be included in any of the above-described formulations as the free acids or bases or as pharmaceutically acceptable salts. Pharmaceutically acceptable salts substantially retain the biologic activity of the free bases.

In some embodiments, the pharmaceutical composition of the present invention further comprises at least one more active ingredient. In other embodiments, a pharmaceutical composition is provided, consisting of a nucleic acid construct of the present invention as an active ingredient.

The nucleic acid constructs of the present invention may be administered to a patient to treat cancer. Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer. The composition can also be administered in therapeutically effective amounts as a portion of an anti-cancer cocktail.

In some embodiments, an expression vector is administered to a patient that comprises a nucleic acid construct of the present invention.

The expression of the nucleic acid molecule may be directed from any suitable promoter and regulated by any appropriate mammalian regulatory element. Promoters may include, but are not limited to, the human cytomegalovirus, simian virus 40, and/or metallothkmein promoters. Enhancers known to preferentially direct gene expression in specific cell types may be used to direct the expression of a nucleic acid. The enhancers used can include, without limitation, those that are characterized as tissue- or cell-specific enhancers. Enhancers are active when placed downstream or upstream from the transcription initiation site. Non-limiting examples of enhancers include the SV40 early gene enhancer, which is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See for example, Enhancers and Eukaryotic Expression, Cold Spring Harbor Press, Cold Spring Harbor, N.Y. 1983.

As discussed herein, it is expected that varying levels of miR-515-5p among individuals affect disease prognosis and therefore affect inter-individual response to treatments. Thus, according to another aspect, the levels of miRNA can be used for personalized medicine. Specifically, the levels of miR-515-5p can be used to determine an effective treatment for a particular subject. Once the particular treatment that will provide the best treatment for the subject is determined, an administration plan can be made.

It is suggested that genetic patterns of miRNAs and their predicted binding sites may have a modifying effect, for example, on the occurrence of or age at diagnosis of breast/ovarian cancer, for example, among Jewish women who carry BRCA1/BRCA2 germline mutations. SNPs associated with miRNA binding sites may be risk factor modifiers in breast/ovarian cancer in ethnically diverse populations of mutation carriers. This may be used as a prognostic tool, as well as a risk assessment tool, for example among BRCA1/2 carriers and other high-risk populations.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### SNP rs28674628 and Cancer Risk

The first example confirms previous findings disclosed in WO 2010/061396 (mentioned above), which was published after the priority date of the present application.

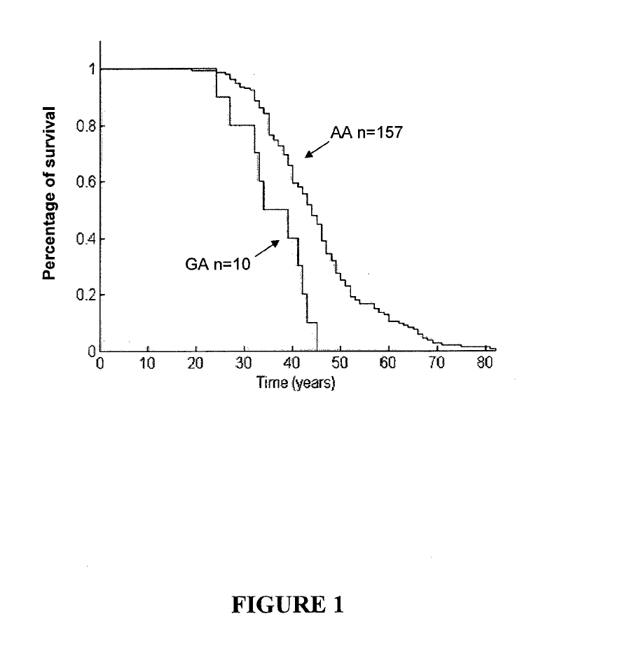

451 Jewish Ashkenazi BRCA1 mutation carriers (390 185delAG and 61 5382InsC mutation carriers) were genotyped for the A/G polymorphism (rs28674628) in the 3′ UTR of the IGF1R gene. Of the study participants, 174 were diagnosed with breast cancer: median age at breast cancer diagnosis was 41.5±11.4 years (mean±SD) (range 19-91 years). Kaplan Meier survival analysis plots showed that the existence of the rare G containing allele was significantly associated with age at diagnosis. All BRCA1 carriers who also carried the G allele of the rs28674628 SNP were diagnosed with breast cancer by age 46 years, whereas almost 50% of the wild type (AA) allele carriers were cancer free at that age (p=0.0003) (FIG. 1).

Participants were recruited from among Jewish women referred for oncogenetic counseling and testing at the Sheba (Tel Hashomer) and Rambam (Haifa) medical centers that were found to harbor one of the predominant BRCA1/2 mutations among Jewish individuals.

Genotyping of the SNPs was carried out using the Sequenom iPLEX™ technology, which is based on the MALDI-TOF platform.

### Example 2

### IGF1R Regulation by miR-515-5p—in Silico Analysis

miRNAs down-regulate the stability and translation of their target mRNAs which posses a conserved complementary sequence to the miRNA ‘seed’ region (nucleotides 2-8 of the miRNA) in their 3′ UTR. A computational prediction based on target predicting algorithms of the inventor (with others)(TargetRank, see Cydney B. Nielsen, Noam Shomron, Rickard Sandberg, Eran Hornstein, Jacob Kitzman, and Christopher B. Burge. RNA. 13: 1-18 (2007)), and other publicly available ones (for example, TargetScan, see www.targetscan.org) has shown that miR-515-5p could bind to the 3′UTR of the IGF1R gene. The computational prediction has also shown that the target area within IGF1R overlaps with SNP rs28674628:

5′TAGATGACTGGTTGCGTCATTTGGAGAAGTGAGTGCTCCTTGATGGTGGAAT 3′ IGF1R
3′GUCUUUCACGAAAGAAAACCUCUU 5' miR-515-5p

The underlined sequences represent the target area within the IGF1R gene and the seed region of miR-515-5p. The boldface characters indicate the location of SNP rs28674628.

### Example 3

### IGF1R Regulation by miR-515-5p—In Vitro Analysis

Methods

Experiments testing the putative functional effect of the different SNP-alleles on IGF1R gene expression levels were performed by measuring reporter gene (Luciferase) activity in cells as a function of the specific SNP appearing in the regulatory region, as well as RNA expression levels using qRT-PCR and protein expression levels using western blot.

In addition, the effect of miR-515-5p over-expression in HeLa-cervical cancer cell line stably transformed with this miR on the endogenous levels of IGF1R versus BRCA was also examined.

Cloning of DNA Fragments into Plasmids:

Cloning of IGF1R 3′UTR bearing either the A allele or the G allele from genomic DNA of patients carrying the desired genotype in the relevant SNP was performed by using a taq proof reading Phusion® Taq Polymerase. The primers were synthesized to include a NotI recognition site at their N terminus and a XhoI recognition site at their C terminus. The primers are listed in Table 1 below. The following PCR reaction was carried out:

| | |
|---|---|
| Step# | PCR reaction |

| 1 | 95° C. | 5 | min |
|---|---|---|---|
| 2 | 94° C. | 30 | sec |
| 3 | 58° C. | 30 | sec |
| 4 | 72° C. | 1.5 | min |
| 5 | Go to #2 30 times | | |
| 6 | 72° C. | 10 | min |

| | | |
|---|---|---|
| 7 | 4° C. | forever |

TABLE 1
primers for cloning:

| SEQ | | |
|---|---|---|
| ID | Primer | |
| NO. | identification | Sequence |
| 6 | IGFIR-XHO- | 5′-TCCCTCGAGCCCATTCCAGCAGTCCCAGTT |
| | FOR | |
| 7 | IGFIR-NOTI- | 5′-ATAAGAATGCGGCCGCTGCCCACGGATGAC |
| | REV | TGCTGAG |

Identification and Separation of DNA in Gel Electrophoresis:

The PCR product was applied to agarose gel electrophoresis under voltage of 100V. Gel concentration was 1% (was determined according to the size of the DNA fragments to be separated: 0.05 Kb-2 KB-2%, 0.5 Kb-10 Kb-1%, 2 Kb-30 Kb-0.5%). EtBr was added to the gel during its preparation to a final concentration of 0.5 μg/ml.

Extraction of the DNA from the Agarose Gel:

The PCR product, 500 bs size, was cut from the gel, frozen in liquid nitrogen for 2 minutes and then thawed at 40° C. until complete thawing was achieved. Next, the fragment was centrifuged for 10 minutes, the supernatant was removed and ethanolic precipitation was performed by adding 3M NaAcet pH 5.2 (1/10 of volume) and 100% ethanol (2-2.5 of volume), mixing and incubating for 15 minutes at −70° C., centrifuging for 10 minutes, washing in 70% ethanol, drying the DNA pellet and re-suspending it in 20 μl DDW.

DNA Restriction with Restriction Enzymes:

The reaction conditions were selected according to the manufacturer instructions, in a final volume of 20 μl:

16-17 μl: DNA (0.5-2 μg)+DDW

1-2 μl: restriction enzymes—10 units

2 μl: 10× reaction buffer

The psiCHECK2 plasmid and PCR products were restricted with restriction enzymes. Alkaline phosphates were added when the plasmid was restricted in order to avoid self-ligation in case of partial digestion.

Ligation:

After additional ethanolic precipitation the restricted plasmid and PCR product were mixed together in a 1:3 molar ratio, in a final volume of 20 μl, for 10 minutes in room temperature:

16-17 μl: DNA (0.5-2 μg)+DDW

1-2 μl: T4 DNA Ligase 1-5 units

2 μl: 10× ligation buffer

Transformation:

Bacteria were thawed on ice. About 100 ng of DNA were added into 70 μl of competent bacteria and incubated on ice for 30 minutes. Next, the competent bacteria were subjected to heat shock at 42° C. for 1 minute, and then they were placed on ice for 2 minutes. 1 ml of LB medium was added to the bacteria and they were incubated for 1 hour at 37° C. with shacking. After the incubation the bacteria were plated on LB plates that contained an appropriate antibiotic.

DNA Extraction from Bacteria:

Extraction of the DNA was performed by using Hi-speed Midiprep kit or Miniprep kit of Qiagen. Sequencing was performed in the Inter-Departmental Research Facility of Tel-Aviv University. Analyses of the DNA and protein sequences were performed by using Vector NTI software which was purchased from Informax.

Cell Cultures Growth:

HEK-293T cells (of human embryonic kidney origin which were transformed) and HeLa cells (of cervical cancer origin), which are comfortable for growth and transfections, were grown in 75 cm² flasks in a DMEM medium supplemented with 10% FBS and P/S antibiotic (Streptomycin 100 mg/ml, Penicillin 100 mg/ml). The medium was replaced every three days and confluent plates were split 1:3. The cells were peeled by Trypsin A, centrifuged for 5 minutes at 500 g at room temperature (RT), re-suspended to achieve separated cells and plated in flasks or in the appropriate experiment plates. The cells were incubated in an incubator at 37° C. in the presence of 5% CO₂

Site-Directed Mutagenesis (SDM):

In the reporter gene experiment, a control vector was used for each of the tested genes, in which the recognition seed of the relevant miR in the 3′UTR sequence was disrupted in order to compare its level of expression to the level of expression from the vectors that include the different SNPs. The preparation of the control vector was performed by using site-directed mutagenesis of three bases/nucleotides in the seed of the recognition sequence in the following way:

QuikChange Site-Directed Mutagenesis Kit (Stratagene) was used. The original plasmid was extracted from E. coli (DAM+) and was therefore methylated. A synthetic, double-stranded plasmid was constructed in a PCR reaction from two homologous primers, which included the desired mutation, on the appropriate DNA template. The primers are listed in Table 2 below. After the PCR reaction the double-stranded DNA was incubated with the enzyme DpnI (which cut the existing methyl groups on the old DNA template but not on the new DNA which included the mutation) for 1 hour at 37° C. At the end of the process only plasmid molecules that were not cut and included the mutation were remained. Next, transformation into bacteria was performed and at the next day DNA was extracted from a single colony and was sent for sequencing to verify the mutation.

TABLE 2
primers for site-directed mutagenesis:

| SEQ | | |
|---|---|---|
| ID | Primer | |
| NO. | identification | Sequence |
| 8 | IGF1RIV-for | 5′-GGTTGCGTCATTTGACTAAGTGAGTGCT |
| | | CCTT |
| 9 | IGF1RIV- | 5′-ATAAGAATGCGGCCGCTGCCCACGGATG |
| | REV | ACTGCTGAG |

Transfection:

The cells were plated in 6 wells plates at a concentration of 1×10̂6 cells/well, or in 12 wells plates at a concentration of 0.5×10̂6 cells/well. On the transfection day: 50-100 μl of the following mixture were added to each well (according to the size of the well): 3 μl LIPOFECTAMINE 2000 and 1 μg DNA in 1 ml medium (after incubation of that mixture for about 30 minutes in RT). Cells were transfected with 10 ng of the expression vector containing the reporter luciferase gene under the 3′UTR control region of the tested gene that was cloned into psiCHECK2, and with 975 ng of the expression vector containing the miRNA-miR-Vec.

Luciferase Assay:

Those assays were performed while inducing over-expression of the miR by using the miR-Vec vector that contains the genomic region of the pri-miRNA under a strong CMV promoter (information about the miR-Vec expression vector can be found, for example, in Voorhoeve et al. (2006) Cell 124, 1169-1181). The following primers were used for cloning miR-515-5p into the vector:

(SEQ ID NO: 10) bamh-mir-515-F:
5′-GCGGATCCGGTACATGCCACCACAGGCG
(SEQ ID NO: 11) RI-mir-515-R:
5′-GCGAATTCGCAGTGAGCAGAGAACATACCA

In order to determine the activity levels of the reporter gene under the 3′UTR of the IGF1R gene, the Dual Luciferase Reporter Assay kit was used. Luminator Ascent (Thermo Electron Corporation) was used to read the results. As detailed above, the 3′UTR of the gene encoding IGF1R, bearing either A or G in the relevant SNP, was fused to the gene encoding the Renilla luciferase. Each result (reading) obtained for the Renilla luciferase was normalized to the result of the Firefly luciferase, an internal control on the same plasmid that neutralizes changes that stem from differences in growth and quality of the cells. According to the different SNP that appears in the 3′UTR, the strength of the binding of the miR-515-5p molecule (that may be endogenous in the cell or inserted by transfection together with the expression vector) to the mRNA product of the fused gene could change, and lead to enhanced or reduced degradation of the mRNA. As a result, an increase or decrease in the measured enzymatic activity may be observed (light emission).

The luciferase assay was performed in 96-wells plates, 24 hours after the transfection to the cells. The indicated quantities are for each well. The growth medium was removed and the cells were washed in 50 ml PBS. 20 ml of PLB (Passive Lysis Buffer) were added for 15 minutes at RT with shacking in order to lyse the cells. 50 ml of LAR II (Luciferase Assay Reagent II) were added and a first measurement of the Firefly luciferase was immediately performed (or control and normalization). 50 ml of Stop&Glow solution were added, that stops the Firefly luciferase activity and at the same time contains substrate for the Renilla luciferase, which reports the activity of the tested gene. A second measurement was performed and the Renilla luciferase results were normalized to the values of the Firefly luciferase.

RNA Extraction from the Cells:

RNA extraction from the cells was performed by using RNA Isolation kit EZ RNA II. The quantities are for 10 cm plates: cells were washed with 2 ml cold PBS, harvested and re-suspended in 2 ml denaturing solution on ice. Phase separation was performed by adding 1.6 ml buffer containing phenol, vortex, adding 0.36 ml buffer containing BCP (1-Bromo-3-Cloropropane), and vortex. Next, 10 minutes incubation at RT was performed followed by 15 minutes centrifugation at 10,000 g to precipitate proteins and DNA, and collection of the upper phase to a fresh tube. 1.2 ml of 100% cold ethanol were added and the solution was incubated over-night at −20° C. At the next day, 50 minutes centrifugation at 14,000 rpm was performed in order to precipitate the RNA, the supernatant was removed and the RNA pellet was washed with 1 ml ethanol 85%. Additional centrifugation for 5 minutes was performed, the rest of the fluids were removed and the pellet was dried. The pellet was re-suspended in 50 ul DDW. The quantity and quality of the obtained RNA was measurement by using NanoDrop.

Reverse Transcription:

By using SuperScript II RT kit:

| | |
|---|---|
| RNA | 8 μl |
| 5x reverse transcription buffer | 4 μl |
| Trehalose 1.7M | 3 μl |
| 10 mM dNTPs mix | 1 μl |
| 0.1M DTT | 2 μl |
| Recombinant Rnasine | 1.5 μl |
| Superscript II RT | 1 μl |
| | Total Vol 20.5 μl |

| | |
|---|---|
| # step | Reverse transcription |

| | | |
|---|---|---|
| 1 | 37° C. | 5 min |
| 2 | 45° C. | 5 min |

Go to #1 5 times

1 ug of the RNA in 4.5 ul volume was heated at 85° C. for two minutes and placed on ice to continue the reverse transcription reaction which was performed by using SuperScript II RT kit:

| | |
|---|---|
| 4.5 μl | RNA (1 ug) |
| 2 μl | 5x reverse transcription buffer |
| 1 μl | Oligo dT* 10 uM |
| 0.5 μl | 10 mM dNTPs mix |
| 1 μl | 0.1M DTT |
| 0.5 μl | Recombinant Rnasine |
| 0.5 μl | Superscript II RT |
| Total Vol 10 μl | |

*RT primer: NTTTTTTTTTTTTTTTTTT

| | |
|---|---|
| # step | Reverse transcription |

| | | | |
|---|---|---|---|
| 1 | 42° c. | 1 | h |
| 2 | 85° c. | 2 | min |

Real-Time PCR with MGB-Taqman® Probe:

Amplification of the cDNA that was obtained after the reverse transcription and diluted to a 0.5 ng/μl concentration, was performed in 96-well plates (ABI) by using a Forward primer which is specific to the tested mature-miRNA, a universal Reverse primer and an MGB-Taqman® probe and PCR mix:

| TaqMan Universal PCR | 10 μl |
|---|---|
| RT-rev-primer-Race 10 μM | 1 μl |
| DDW | 6 μl |
| Mix at room temp and place on ice | |
| cDNA 0.5 ng/μ | 1 μl |
| Mature-mir-Fwd Primer 10 μM | 1 μl |
| MGB-miR probe 5 μM | 1 μl |
| | Total Vol 20 μl |

Real-Time PCR with Non-Specific SYBR Probe:

Amplification of the cDNA, that was obtained after the reverse transcription and diluted to a 0.5 ng/μl concentration, was performed in 96-well plates (ABI) by using Forward and Reverse primers which are specific to the miRNA hairpin or the tested miRNA and a non-specific probe. Power SYBR Green PCR master Mix:

| | |
|---|---|
| cDNA 0.5 ng/μ | 8 μl |
| SYBR Green | 10 μl |
| Rev-primer 10 μM | 1 μl |
| Fwd-primer 10 μM | 1 μl |
| DDW | 6 μl |
| | Total Vol 26 μl |

The PCR program was the same for the two probes:

| | |
|---|---|
| # step | Real-Time PCR |

| | | | |
|---|---|---|---|
| 1 | 95° c. | 10 | min |
| 2 | 95° c. | 15 | sec |
| 3 | 60° c. | 1 | min |

Go to #2 42 times

Amplification was performed in an ABI 7900 system instrument (Applied Biosystems). The Ct (Cycle threshold) values that were obtained were normalized according to a control of other known miRNAs or of known House-keeping genes. Significance of the results was calculated by using bi-lateral uncoupled t-test.

Results

A. Interaction Between IGF1R 3′UTR and miR-515-5p In Vitro

The in silico prediction was experimentally confirmed by the finding that Luciferase activity is lower when expressed under a cloned 3′ UTR regulatory region of IGF1R with AA genotype, compared to GA genotype and ACT knockdown (the binding site of the miRNA), during miR-515-5p over-expression in HEK and HeLa cells.

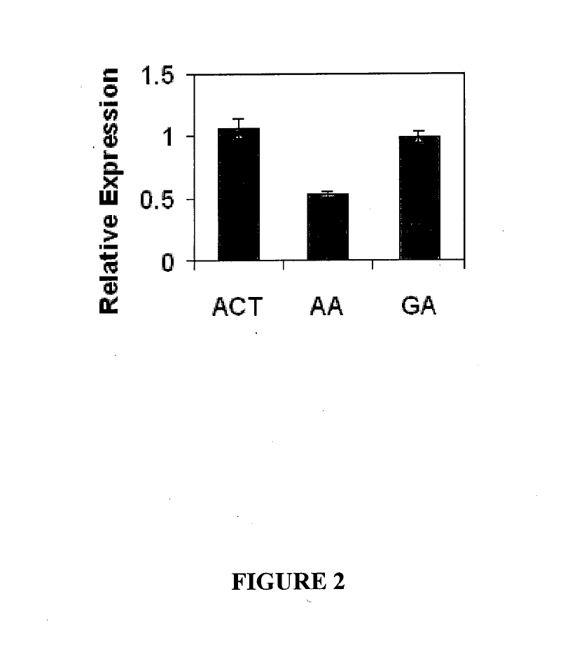

A dual Luciferase assay in HEK-293T cells co-transfected with miR-515-5p and a psiCHECK2 vector containing either a construct of the 3′UTR IGF1R homozygote (AA), heterozygote (AG) or a knowkdown control (ACT), confirmed that miR-515-5p over-expression can inhibit the expression of IGF1R (FIG. 2).

B. Regulation of Endogenous IGF1R Versus BRCA in HeLa Cells

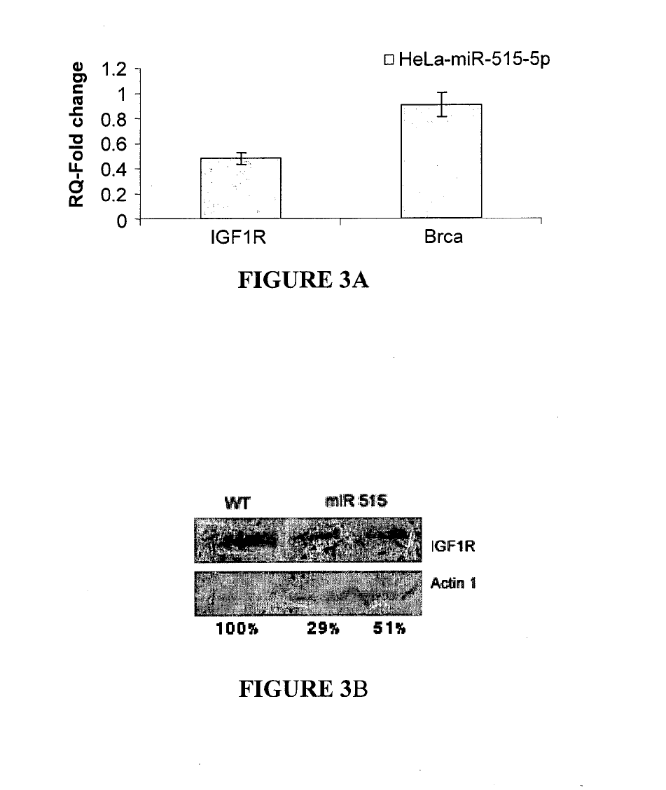

Further support to the ability of miR-515-5p to inhibit the expression of IGF1R was obtained by measuring the expression of the endogenous IGF1R or BRCA genes in HeLa cells stably transformed with miR-515-5p. The analysis showed that over-expression of miR-515-5p reduces IGF1R expression and not BRCA expression. The analysis was performed by quantitative real time RT-PCR (qRT-PCR), detected as a change in expression (FIG. 3A).

The analysis also showed that the levels of the IGF1R protein were reduced in the presence of miR-515-5p (FIG. 3B). Two different HeLa cell culture plates were stably transformed with miR-515-5p. The large amount of miR-515-5p reduced the levels of IGF1R protein to 51% in one case and to 29% in another case, as indicated below each lane of FIG. 3B. The difference might be due to different levels of miRNA expression in the two cases.

IGF1R protein level was quantified by using a Western blot assay and the Rabbit polyclonal anti-IGF1R antibody (Santa Cruz Biotechnology) using standard procedures for blotting. The normalization was performed with mouse monoclonal anti-actin antibody (Sigma).

These results suggest that over-expression of miR-515-5p can reduce the expression level of the IGF1R gene and protein.

### Example 4

### Analysis of Breast Tissues Derived from Breast Cancer Patients

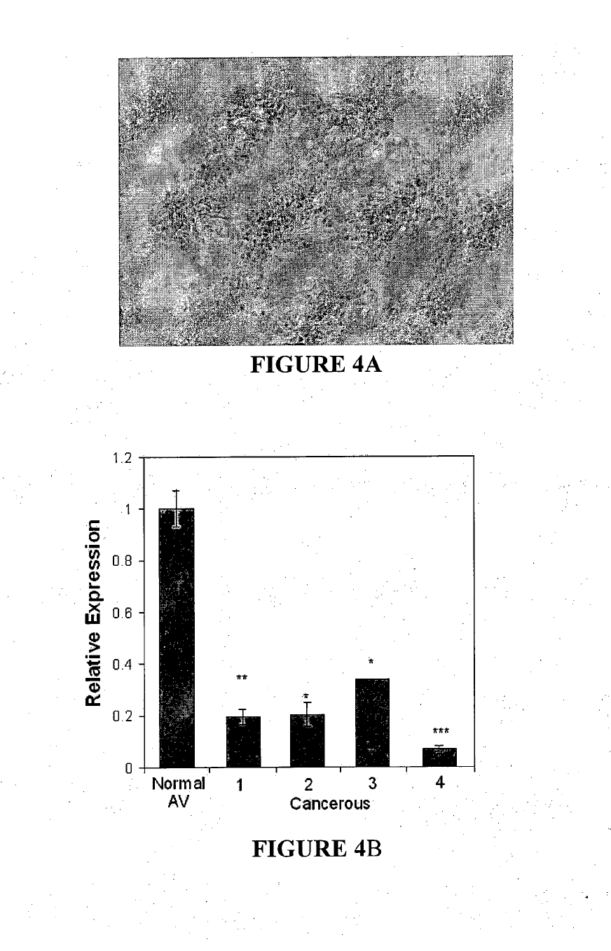

In order to evaluate to what extent the identified interaction has any clinical relevance, and in order to decipher the putative role that miR-515 has in vivo, the quantity of miR-515-5p and mRNA levels of IGF1R was determined somatically in breast tissues derived from breast cancer patients BRCA1/BRCA2 mutation carriers, comparing the levels in tumors and adjacent non-tumorous tissues. Additionally, immunohistochemistry was used to assess protein expression of IGF1R somatically in the same patients. Immunostaining for IGF1R was found to be positive in carcinoma cells in contrast to negative non-neoplastic surrounding tissue (FIG. 4A). A substantial difference in miR-515-5p expression levels favoring normal tissue was noted (FIG. 4B), indicating that miR-515-5p is down regulated in breast cancer tissue to relieve the cells from low IGF1R levels.

### Example 5

### miR-515-5p Serum Levels in Healthy Subjects Versus Cancer Patients

Serum samples from healthy subjects and from breast or ovarian cancer patients were subjected to real-time PCR to evaluate the level of miR-515-5p in the serum. The Ct values (cycle threshold) of the different samples were compared. The results showed that Ct values of samples from healthy subjects typically ranged from 20-30, or from 20-35, whereas Ct values of samples from cancer patients were above 35, or even above 50, indicating a lower level of miR-515-5p in the samples from cancer patients.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for cancer diagnosis, prognosis or monitoring in a subject, the method comprising:
(i) providing a biological sample from the subject;
(ii) measuring the level of miR-515-5p in the sample;
(iii) comparing the level of miR-515-5p in the sample to a control level of miR-515-5p;
wherein a significantly decreased miR-515-5p level relative to the control is indicative of the subject having cancer, or being susceptible to developing cancer, or having cancer associated with poor prognosis.

2. The method of claim 1, wherein the biological sample is a bodily fluid.

3. The method of claim 2, wherein the bodily fluid is selected from the group consisting of serum, plasma and blood.

4. The method of claim 1, wherein the biological sample is a tissue.

5. The method of claim 4, wherein the biological sample is a tissue comprising cancer cells or suspected of harboring cancer cells.

6. The method of claim 1, wherein cancer diagnosis in a subject comprises:
(i) providing a sample comprising serum from the subject;
(ii) measuring the serum level of miR-515-5p in the sample;
(iii) comparing the serum levels of miR-515-5p in the sample to a control serum level of miR-515-5p;
wherein a significantly decreased miR-515-5p serum level relative to the control is indicative of the subject having, or being susceptible to developing cancer.

7. The method of claim 1, wherein cancer prognosis in a subject comprises:
(i) providing a biological sample from the subject;
(ii) measuring the level of miR-515-5p in the sample;
(iii) comparing the level of miR-515-5p in the sample to a control level of miR-515-5p;
wherein a significantly decreased miR-515-5p level relative to the control is indicative of poor cancer prognosis.

8. The method of claim 7, wherein the biological sample is selected from the group consisting of serum, plasma and blood.

9. The method of claim 7, wherein the biological sample comprises cancer cells from the subject.

10. The method of claim 1, wherein monitoring cancer treatment in a subject comprises:
(i) providing a biological sample from the subject;
(ii) measuring the level of miR-515-5p in the sample;
(iii) comparing the resulting level of miR-515-5p in the sample to initial levels of miR-515-5p in a sample from the same subject;
wherein an increase in the level of miR-515-5p compared to the initial levels is indicative of a positive response to treatment.

11. The method of claim 10, wherein the biological sample is selected from the group consisting of serum, plasma and blood.

12. The method of claim 10, wherein the biological sample comprises cancer cells.

13. The method of claim 1, wherein the cancer type is characterized by at least one of increased level of IGF1R and decreased level of miR-515-5p in at least a portion of the cancer cells compared to normal levels in non-cancerous cells.

14. The method of claim 13, wherein the cancer type is selected from the group consisting of breast, ovarian, prostate, lung, pancreas, hepatocellular carcinoma and colorectal.

15. The method of claim 14, wherein the cancer type is selected from the group consisting of breast and ovarian cancer.

16. The method of claim 15, wherein the subject carries at least one mutation selected from the group consisting of BRCA1 mutation and BRCA2 mutation.

17. The method of claim 1, wherein the subject carries an AA genotype in a polymorphic site within the IGF1R gene having a reference sequence (rs) number 28674628.

18. A method for treating cancer, the method comprising administering to a subject in need thereof a nucleic acid construct comprising miR-515-5p or an analogue or derivative thereof.

19. The method of claim 18, wherein the analogue or derivative comprises the sequence set forth in SEQ ID NO: 4.

20. The method of claim 18, wherein the cancer type is characterized by at least one of increased level of IGF1R and decreased level of miR-515-5p in at least a portion of the cancer cells compared to normal levels in non-cancerous cells.

21. The method of claim 20, wherein the cancer type is selected from the group consisting of breast, ovarian, prostate, lung, pancreas, hepatocellular carcinoma and colorectal.

22. The method of claim 21, wherein, the cancer type is selected from the group consisting of breast and ovarian cancer.

23. The method of claim 22, wherein the subject carries at least one mutation selected from the group consisting of BRCA1 mutation and BRCA2 mutation.

24. The method of claim 18, wherein the nucleic acid construct is administered in an amount effective for suppression of IGF1R cellular expression.

25. The method of claim 18, wherein the subject carries an AA genotype in the polymorphic site within the IGF1R gene designated by reference sequence (rs) number 28674628.

26. The method of claim 18, wherein the nucleic acid construct comprises a precursor of miR-515-5p or an analogue or derivative thereof.

27. The method of claim 18, wherein the nucleic acid construct is formulated into a pharmaceutical composition.

28. The method of claim 18, further comprising a step of measuring at least one parameter selected from the group consisting of the level of IGF1R and the level of miR-515-5p in cancer cells obtained from the subject to be treated prior to the beginning of the treatment.

29. The method of claim 18, further comprising periodical measurements of at least one parameter selected from the group consisting of the level of IGF1R and the level of miR-515-5p in cancer cells obtained from the subject.

30. A method for treating cancer, the method comprising administering to a subject in need thereof a nucleic acid construct comprising a sequence encoding miR-515-5p or an analogue or derivative thereof.

31. The method of claim 30, wherein the nucleic acid construct comprising a sequence encoding miR-515-5p or an analogue or derivative thereof is an expression vector.

32. A nucleic acid construct comprising miR-515-5p or an analogue or derivative thereof, for use in the treatment of cancer.

33. A nucleic acid construct comprising a sequence encoding miR-515-5p or an analogue or derivative thereof, for use in the treatment of cancer.

34. A composition comprising a nucleic acid construct comprising miR-515-5p or an analogue or derivative thereof, for use in the treatment of cancer.

35. A composition comprising a nucleic acid construct comprising a sequence encoding miR-515-5p or an analogue or derivative thereof, for use in the treatment of cancer.

36. A method of personalized medicine for cancer, the method comprising measuring cellular levels of miR-515-5p in cancer cells from a subject having a particular cancer, and proposing a treatment with a likelihood of being effective for said subject based on the cellular levels of miR-515-5p in said cancer cells.

37. The method of claim 36, wherein the resulting levels of miR-515-5p are compared to levels of miR-515-5p determined in a previous measurement from the same subject.

38. A method of inhibiting cancer cells proliferation comprising manipulating the levels of miR-515-5p in the cancer cells.

39. The method of claim 38, wherein over-expression of miR-515-5p is induced in the cancer cells.

40. The method of claim 39, wherein the method comprising administering a nucleic acid construct comprising miR-515-5p or an analogue or derivative thereof.

41. The method of claim 39, wherein the method comprising administering a nucleic acid construct encoding a sequence comprising miR-515-5p or an analogue or derivative thereof.

42. The method of claim 39, wherein the cancer cells harbor an AA genotype in the polymorphic site within the IGF1R gene designated by reference sequence (rs) number 28674628.
